SEQUENCE LISTING (8 sequences)

SEQ ID NO: 1 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 2 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 3 - DNA, 657 bp, Clostridium acetobutylicum
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 4 - protein, 218 aa, Clostridium acetobutylicum
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 5 - DNA, 666 bp, Clostridium acetobutylicum
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 6 - protein, 221 aa, Clostridium acetobutylicum
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 7 - DNA, 1056 bp, Clostridium beijerinckii
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 8 - protein, 351 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

(19) United States
(12) Patent Application Publication
(10) Pub. No.: US 2018/0148743 A1
(43) Pub. Date: May 31, 2018

(54) PRODUCTION OF 3-HYDROXYBUTYRATE

(71) Applicant: EVONIK DEGUSSA GMBH, Essen (DE)

(72) Inventors: Thomas HAAS, Münster (DE); Anja HECKER, Münster (DE); Markus PÖTTER, Münster (DE); Thomas BÜLTER, Duisburg (DE)

(73) Assignee: EVONIK DEGUSSA GMBH, Essen (DE)

(21) Appl. No.: 15/578,331

(22) PCT Filed: Jul. 15, 2016
(86) PCT No.: PCT/EP2016/066875

§ 371 Date: Nov. 30, 2017

(30) Foreign Application Priority Data
Jul. 29, 2015 (EP) 15178769.4

(51) Int. Cl. C12P 7/42; C12N 9/04; C12N 15/52; C12N 9/10

(52) U.S. Cl. CPC C12P 7/42; C12Y 401/01004; C12Y 208/03008; C12Y 101/01001; C12N 9/0006; C12N 15/52; C12N 9/1029; C12Y 203/01009

(57) ABSTRACT

There is provided amicrobial cell which is capable of producing acetoacetate, 3-hydroxybutyrate and/or 3-hydroxybutyrate variants, wherein the cell is genetically modified to comprise an increased expression relative to its wild type cell of: an enzyme E₁ capable of catalysing the conversion of acetyl-CoA to acetoacetyl-CoA; an enzyme E₂ capable of catalysing the conversion of acetoacetyl-CoA to acetoacetate; and an enzyme E₃ capable of catalysing the conversion of acetoacetate to 3-hydroxybutyrate and/or variants thereof.

### FIELD OF THE INVENTION

The present invention relates to a biotechnological method of producing 3-hydroxybutyrate and/or variants thereof. In particular, the method relates to a biotechnological production of 3-hydroxybutyrate from a carbon source via acetoacetyl-CoA and acetoacetate.

### BACKGROUND OF THE INVENTION

3-hydroxybutyrate also known as beta-hydroxybutyric acid is an organic compound with the formula CH₃CH(OH)CH₂CO₂H. It is a beta hydroxy acid and is a chiral compound having two enantiomers, D-3-hydroxybutyric acid and L-3-hydroxybutyric acid. Its oxidized and polymeric derivatives occur widely in nature. 3-hydroxybutyrate is the precursor to polyesters, which are biodegradable plastics including poly(3-hydroxybutyrate). 3-hydroxybutyrate is also the precursor for production of 2-hydroxyisobutyric acid and polyhydroxyalkanoates containing 2-hydroxyisobutyric acid monomer units including methacrylic acid. Methacrylic acid, its esters and polymers are widely used for producing acrylic glass panes, injection-moulded products, coatings and many other products.

There are several methods known in the art for production of 3-hydroxybutyrate. However, most of these methods use petroleum as a starting point for production of 3-hydroxybutyrate. The over-utilization of petroleum and petroleum based products has caused environmental issues including increasing atmospheric concentration of CO₂, pollution from petrochemical production and use, and disposal of non-biodegradable plastic materials. More importantly, petroleum resources are finite and not renewable in nature. For these reasons it is necessary to seek alternative approaches to produce fuels and chemicals using renewable resources. Photosynthetic cyanobacteria have attracted significant attention in recent years as a ‘microbial factory’ to produce biofuels and chemicals due to their capability to utilize solar energy and CO₂ as the sole energy and carbon sources, respectively. Cyanobacteria are also natural producers of the naturally-occurring biodegradable plastic poly-hydroxybutyrate (PHB). Despite efforts to enhance PHB biosynthesis through both genetic engineering strategies and the optimization of culture conditions, PHB biosynthesis by cyanobacteria was a multistage cultivation process that involved nitrogen starvation followed by supplementation of fructose or acetate, which does not capitalize on the important photosynthetic potential of cyanobacteria. Most importantly, as neither lipids nor PHB are secreted by the cells, the required processes for their extraction are energy-intensive and remain as one of the major hurdles for commercial applications.

Accordingly, there is a need in the art for a more efficient and/or an alternative biotechnological method for producing 3-hydroxybutyrate.

### DESCRIPTION OF THE INVENTION

The present invention provides a cell that has been genetically modified to produce acetoacetate. In particular, the cell may be capable of converting the acetoacetate to 3-hydroxybutyrate. This is advantageous as a single cell may be used to produce 3-hydroxybutyrate, making the process simpler and requiring no steps of separation and thus no loss of material along the way. The cell according to any aspect of the present invention may also be reused reducing waste and causing an overall increased efficiency of production of 3-hydroxybutyrate. 3-hydroxybutyrate may also be produced from waste gases thus not relying on fossil fuels.

According to one aspect of the present invention, there is provided a microbial cell which is capable of producing 3-hydroxybutyrate and/or variants thereof, wherein the cell is genetically modified to comprise an increased expression relative to its wild type cell of:
an enzyme E₁ capable of catalysing the conversion of acetyl-CoA to acetoacetyl-CoA;
an enzyme E₂ capable of catalysing the conversion of acetoacetyl-CoA to acetoacetate; and
an enzyme E₃ capable of catalysing the conversion of acetoacetate to 3-hydroxybutyrate and/or variants thereof with the proviso that the genetically modified cell has reduced or no expression of acetoacetate decarboxylase (adc; EC 4.1.1.4).

The phrase “wild type” as used herein in conjunction with a cell or microorganism may denote a cell with a genome make-up that is in a form as seen naturally in the wild. The term may be applicable for both the whole cell and for individual genes. The term “wild type” therefore does not include such cells or such genes where the gene sequences have been altered at least partially by man using recombinant methods.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more acetoacetate and/or 3-hydroxybutyrate than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (3-hydroxybutyrate) in the nutrient medium.

The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce acetoacetate and/or 3-hydroxybutyrate and/or variants thereof. The wild type microorganism relative to the genetically modified microorganism of the present invention may have none or no detectable activity of the enzymes that enable the genetically modified microorganism to produce the acetoacetate and/or 3-hydroxybutyrate and/or variants thereof. As used herein, the term ‘genetically modified microorganism’ may be used interchangeably with the term ‘genetically modified cell’. The genetic modification according to any aspect of the present invention is carried out on the cell of the microorganism.

The cells according to any aspect of the present invention are genetically transformed according to any method known in the art. In particular, the cells may be produced according to the method disclosed in WO/2009/077461.

The phrase ‘the genetically modified cell has an increased activity, in comparison with its wild type, in enzymes’ as used herein refers to the activity of the respective enzyme that is increased by a factor of at least 2, in particular of at least 10, more in particular of at least 100, yet more in particular of at least 1000 and even more in particular of at least 10000. In one example, the increased expression of an enzyme according to any aspect of the present invention may be 5, 10, 15, 20, 25, 30, 25, 40, 45, 50, 55, 60, 65, 70, 75, 80, 85, 90, 95 or 100% more relative to the expression of the enzyme in the wild type cell. Similarly, the decreased expression of an enzyme according to any aspect of the present invention may be 5, 10, 15, 20, 25, 30, 25, 40, 45, 50, 55, 60, 65, 70, 75, 80, 85, 90, 95 or 100% less relative to the expression of the enzyme in the wild type cell.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. In one example, ‘increased activity of an enzyme’ in a cell may refer to an overexpression of the enzyme in the cell.

The microorganism according to any aspect of the present invention may be an acetogenic bacterium. The term “acetogenic bacteria” as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway. In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui).

More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ERI2, Clostridium ljungdahlii COL and Clostridium ljungdahlii O-52 may be used in the conversion of synthesis gas to hexanoic acid. These strains for example are described in WO 98/00558, WO 00/68407, ATCC 49587, ATCC 55988 and ATCC 55989.

In another example, the strain Clostridium autoethanogenum (DSM 10061, DSM 19630 and DSM 23693) may be used. In particular, the strain may be Clostridium autoethanogenum DSM 10061.

3-hydroxybutyrate (3HB) and variants thereof include but are not limited to (S)-3-hydroxybutyric acid, (S)-3-hydroxybutyrate ester, (R)-3-hydroxybutyric acid, (R)-3-hydroxybutyrate ester, sodium 3-hydroxybutyrate, methyl 3-hydroxybutyrate and the like. In particular, variants of 3-HB are selected from the group consisting of (S)-3-hydroxybutyric acid, (S)-3-hydroxybutyrate ester, (R)-3-hydroxybutyric acid, (R)-3-hydroxybutyrate ester, sodium 3-hydroxybutyrate, and methyl 3-hydroxybutyrate.

The cell according to any aspect of the present invention, may produce at least acetoacetate. The acetoacetate may then be converted to 3HB. However, the final result always comprises a small percentage of acetoacetate that has not been converted to 3HB. Accordingly, the cell according to any aspect of the present invention may be capable of producing acetoacetate and/or 3HB.

The cell according to any aspect of the present invention, wherein E₁ may be a thiolase (E.C.2.3.1.9), E₂ may be an acetoacetate CoA transferase (EC 2.8.3.8) and/or E₃ may be a secondary alcohol dehydrogenase (EC 1.1.1.1). The combination of these enzymes in the cell according to any aspect of the present invention allows for a ‘pull’ towards the formation of the final desired product, 3-hydroxybutyrate and/or variants thereof from the starting carbon source. In particular, the cell according any aspect of the present invention may lead to the hydrolysis of acetoacetyl-CoA with a high reaction rate at low substrate concentrations and therefore can prevent accumulation of acetoacetyl-CoA and establish a “pull” on the preceding, thiolase mediated reversible acetoacetyl-CoA biosynthesis reaction.

In particular, E₁ may be capable of catalyzing the conversion of acetyl-CoA to acetoacetyl-CoA. E₁ may be an acetoacetyl-CoA thiolase also known as an acetyl-Coenzyme A acetyltransferase. Acetoacetyl-CoA thiolase enzymes include the gene products of atoB from E. coli (Martin et al., 2003) with accession number NP_416728, thiolase derived from C. acetobutylicum. More in particular, E₁ may comprise an amino acid sequence that has 50, 55, 60, 65, 70, 75, 80, 85, 90, 95 or 100% sequence identity to SEQ ID NO: 2. Even more in particular, the cell according to any aspect of the present invention may be genetically modified to comprise the sequence of SEQ ID NO:2.

A skilled person may be capable of identifying other thiolases that may play the role of E₁. In particular, the a skilled person may be capable of assessing whether a functionally equivalent variant has substantially the same function as the nucleic acid or polypeptide of which it is a variant using any number of known methods. In one example, the methods outlined in Wiesenborn et al 1988, Wiesenborn et al, 1989, Peterson and Bennet, 1990, Ismail et al., 1993, de la Plaza et al, 2004 or may be used to assess the enzyme activity of E₁.

E₂ may be an acetoacetate CoA transferase (EC 2.8.3.9). The acetoacetate CoA transferase conserves the energy stored in the CoA-ester bond. These enzymes either naturally exhibit the desired acetoacetyl-CoA transferase activity or they can be engineered via directed evolution to accept acetetoacetyl-CoA as a substrate with increased efficiency. In particular, such enzymes, may also be capable of catalyzing the conversion of 3-hydroxybutyryl-CoA to 3-hydroxybutyrate via a transferase mechanism. Examples of E₂ may include the CoA transferase from E. coli with accession number P76459.1 or P76458.1 (Hanai et al., 2007), ctfAB from C. acetobutylicum with accession number NP_149326.1 or NP_149327.1 (Jojima et al., 2008), ctfAB from Clostridium saccharoperbutylacetonicum with accession number AAP42564.1 or AAP42565.1 (Kosaka et al., 2007) and the like. In particular, E₂ may also be selected from the group consisting of the gene products of cat1, cat2, and cat3 of Clostridium kluyveri with accession number P38946.1, P38942.2, and EDK35586.1 respectively (Seedorf et al., 2008; Sohling and Gottschalk, 1996), transferase products of Trichomonas vaginalis with accession number XP_001330176 (van Grinsven et al., 2008), Trypanosome brucei with accession number XP_828352 (Riviere et al, 2004), Fusobacterium nucleatum (Barker et al., 1982), Clostridium SB4 (Barker et al., 1978), Clostridium acetobutylicum (Wiesenborn et al., 1989), FN0272 and FN0273 with accession numbers NP_603179.1 and NP_603180.1 respectively (Kapatral et al., 2002), homologs in Fusobacterium nucleatum such as FN1857 and FN1856 with accession numbers NP_602657.1 and NP_602656.1 (Kreimeyer, et al., 2007), transferase products of Porphyrmonas gingivalis with accession number NP_905281.1 or NP_905290.1 and Thermoanaerobacter tengcongensis with accession number NP_622378.1 or NP_622379.1 (Kreimeyer, et al., 2007). More in particular, E₂may comprise an amino acid sequence that has 50, 55, 60, 65, 70, 75, 80, 85, 90, 95 or 100% sequence identity to SEQ ID NO: 4 or 6. In particular, E₂ may comprise an amino acid sequence SEQ ID NO: 4 and 6. E₂ may comprise a nucleotide sequence that has 50, 55, 60, 65, 70, 75, 80, 85, 90, 95 or 100% sequence identity to SEQ ID NO: 3 or 5. More in particular, E₂ may comprise a nucleotide sequence that has 50, 55, 60, 65, 70, 75, 80, 85, 90, 95 or 100% sequence identity to SEQ ID NO: 3 and 5.

The expression of E₂ may be measured using any method known in the art. In particular, the increase in expression of E₂ may be measured by determining the amount of final product obtained in the presence of the enzyme and comparing the result to the amount of final product obtained in the absence of the enzyme E₂. In one example the final product may be a 3-hydroxybutyrate. In another example, the expression of E₂ may be determined by determining the amount of E₂ protein expressed in the resulting medium. In one example the expression of E₂ may be measured using the method disclosed in Charrier C., 2006.

E₃ may be an alcohol dehydrogenase. “Alcohol dehydrogenases” may include alcohol dehydrogenases which are capable of catalysing the conversion of ketones (such as acetone) to secondary alcohols (such as isopropanol), or vice versa. Such alcohol dehydrogenases include secondary alcohol dehydrogenases and primary alcohol dehydrogenases. A “secondary alcohol dehydrogenase” is one which can convert ketones (such as acetone) to secondary alcohols (such as isopropanol), or vice versa. A “primary alcohol dehydrogenase” is one which can convert aldehydes to primary alcohols, or vice versa; however, a number of primary alcohol dehydrogenases are also capable of catalysing the conversion of ketones to secondary alcohols, or vice versa. These alcohol dehydrogenases may also be referred to as “primary-secondary alcohol dehydrogenases”. Membrane-bound, flavin-dependent alcohol dehydrogenases of the Pseudomonas putida GPO1 AlkJ type exist which use flavor cofactors instead of NAD+. A further group comprises iron-containing, oxygen-sensitive alcohol dehydrogenases which are found in bacteria and in inactive form in yeast. Another group comprises NAD+-dependent alcohol dehydrogenases, including zinc-containing alcohol dehydrogenases, in which the active center has a cysteine-coordinated zinc atom, which fixes the alcohol substrate. In one example, under the expression “alcohol dehydrogenase”, as used herein, it is understood to mean an enzyme which oxidizes an aldehyde or ketone to the corresponding primary or secondary alcohol. In particular, the alcohol dehydrogenase according to any aspect of the present invention may be an NAD+-dependent alcohol dehydrogenase, i.e. an alcohol dehydrogenase which uses NAD+ as a cofactor for oxidation of the alcohol or NADH for reduction of the corresponding aldehyde or ketone. In the most preferred embodiment, the alcohol dehydrogenase is an NAD+-dependent, zinc-containing alcohol dehydrogenase. Examples of suitable NAD+-dependent alcohol dehydrogenases may include the alcohol dehydrogenase A from Rhodococcus ruber (database code AJ491307.1) or a variant thereof. Further examples comprising the alcohol dehydrogenases of Ralstonia eutropha (ACB78191.1), Lactobacillus brevis (YP_795183.1), Lactobacillus kefiri (ACF95832.1), from horse liver, of Paracoccus pantotrophus (ACB78182.1) and Sphingobium yanoikuyae (EU427523.1) and also the respective variants thereof. In one example, the expression “NAD(P)+-dependent alcohol dehydrogenase”, as used herein, designates an alcohol dehydrogenase which is NAD+- and/or NADP+-dependent.

In one example, E₃ may be a secondary alcohol dehydrogenase or selected from other alcohol dehydrogenases or equivalently aldehyde reductases and can also serve as candidates for 3-hydroxybutyraldehyde reductase. T. E₃ may be the product of the gene selected from the group consisting of adhI from Geobacillus thermoglucosidasius with accession number AAR91477.1 (Jeon et al., 2008), SADH from C. beijerinckii the alcohol dehydrogenases disclosed in Tani et al., 2000 with accession number BAB122273.1 may be used as E₃. E₃ may also be selected from the group consisting of ADH2 from Saccharomyces cerevisiae with accession number NP_014032.1 (Atsumi et al., 2008), yqhD from E. coli with accession number NP_417484.1 (Sulzenbacher et al., 2004 and Perez et al., 2008), bdh I and bdh II from C. acetobutylicum with accession number NP_349892.1 and NP_349891.1 respectively (Walter et al.,1992), and ADH1 from Zymomonas mobilis with accession number YP_162971.1 (Kinoshita et al., 1985).

More in particular, E₃ may be a secondary alcohol dehydrogenase. Even more in particular, E₃ may be a secondary alcohol dehydrogenase from C. beijerinckii. E₃ may comprise an amino acid sequence that has 50, 55, 60, 65, 70, 75, 80, 85, 90, 95 or 100% sequence identity to SEQ ID NO: 8. E₃ may comprise a nucleotide sequence that has 50, 55, 60, 65, 70, 75, 80, 85, 90, 95 or 100% sequence identity to SEQ ID NO: 7.

Any accession number used in the application refers to the respective sequence from the Genbank database run by the NCBI, wherein the release referred to is the one available online on the 30 Mar. 2015.

The expression of E₃ may be measured using any method known in the art. In particular, the increase in expression of E₃ may be measured by determining the amount of final product obtained in the presence of the enzyme and comparing the result to the amount of final product obtained in the absence of the enzyme E₃. In one example the final product may be a primary and/or secondary alcohol. In another example, the expression of E₃ may be determined by determining the amount of E₃ protein expressed in the resulting medium. In one example the expression of E₃ may be measured using the method disclosed in Ismaiel, A. A. (1993).

According to any aspect of the present invention, the cell may comprise enzymes E₁, E₂ and E₃. In particular, E₁ may be a thiolase (E.C.2.3.1.9), E₂ may be an acetoacetate CoA transferase and E₃ may be a secondary alcohol dehydrogenase. More in particular, E₁ may comprise amino acid sequence SEQ ID NO:2, E₂ may comprise amino acid sequence SEQ ID NO:4 or 6, and E₃ may comprise amino acid sequence SEQ ID NO:8. Even more in particular, E₁ may comprise amino acid sequence SEQ ID NO:2, E₂ may comprise amino acid sequence SEQ ID NO:4 and 6, and E₃ may comprise amino acid sequence SEQ ID NO:8. In one example, E₁ may consists of amino acid sequence SEQ ID NO:2, E₂ may consists of amino acid sequence SEQ ID NO:4 and 6, and E₃ may consists of amino acid sequence SEQ ID NO:8.

According to any aspect of the present invention, the cell may comprise enzymes E₁, E₂ and E₃ and reduced or no expression of acetoacetate decarboxylase (Adc; EC 4.1.1.4). The cell according to any aspect of the invention, in order to express significantly reduced levels of adc, may be genetically modified to remove expression of adc which may be achieved by using standard recombinant DNA technology known to the person skilled in the art. The gene sequences respectively responsible for production of adc may be inactivated or partially or entirely eliminated. Thus, the cell according to any aspect of the present invention expresses reduced or undetectable levels of adc or expresses functionally inactive adc.

In one example, the cell according to any aspect of the present invention may express adc naturally and may be genetically modified to reduce the expression of adc in the cell to about 0% or undetectable levels relative to the wild type cell. In another example, the cell according to any aspect of the present invention has about 0% or undetectable levels of expression of adc in its' wild type form. In particular, when the cell according to any aspect of the present invention has undetectable expression of the enzyme adc, the activity of the secondary dehydrogenase (E₃) may be enhanced thus possibly resulting in increased production of acetoacetate and/or 3-hydroxybutyrate. The final product, acetoacetate and/or 3-hydroxybutyrate may also be found in the reaction mixture. Thus the cell according to any aspect of the present invention may result in the export and/or release of acetoacetate and/or 3-hydroxybutyrate into the aqueous medium. The cells will thus not have to go through a further step of extracting the target products and therefore possibly killing the cells in the process of doing so.

Enzymes that are encoded by nucleic acids that have 90%, 95%, 99% and in particular 100% identity to the sequences according to SEQ ID NOs: 1, 3, 5 and 7, are suitable in the method of the present invention. The “nucleotide identity” relative to SEQ ID NOs: 1, 3, 5 and 7 is determined using known methods. In general, special computer programs with algorithms are used, taking into account special requirements. Methods that may be used for determination of identity first produce the greatest agreement between the sequences to be compared. Computer programs for determination of identity comprise, but are not restricted to, the GCG software package, including GAP (Deveroy, J. et al., 1984), and BLASTP, BLASTN and FASTA (Altschul, S. et al., 1990). The BLAST program can be obtained from the National Center for Biotechnology Information (NCBI) and from other sources (BLAST Manual, Altschul S. et al., 1990).

The well-known Smith-Waterman algorithm can also be used for determining nucleotide identity.

Parameters for nucleotide comparison may comprise the following:
Algorithm Needleman and Wunsch, 1970,
Comparison matrix
   Matches=+10
   Mismatches=0
   Gap penalty=50
   Gap length penalty=3

The GAP program is also suitable for use with the parameters given above. These parameters are usually the default parameters in the nucleotide sequence comparison.

The culture medium to be used must be suitable for the requirements of the particular strains. Descriptions of culture media for various microorganisms are given in “Manual of Methods for General Bacteriology”.

All percentages (%) are, unless otherwise specified, mass percent.

With respect to the source of substrates comprising carbon dioxide and/or carbon monoxide, a skilled person would understand that many possible sources for the provision of CO and/or CO₂ as a carbon source exist. It can be seen that in practice, as the carbon source of the present invention any gas or any gas mixture can be used which is able to supply the microorganisms with sufficient amounts of carbon, so that acetate and/or ethanol, may be formed from the source of CO and/or CO₂.

Generally for the cell of the present invention the carbon source comprises at least 50% by weight, at least 70% by weight, particularly at least 90% by weight of CO₂ and/or CO, wherein the percentages by weight-% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

According to any aspect of the present invention, the carbon source may be synthesis gas. Synthesis gas can for example be produced as a by-product of coal gasification. Accordingly, the microorganism according to any aspect of the present invention may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

A skilled person would understand the other conditions necessary to carry out the method of the present invention. In particular, the conditions in the container (e.g. fermenter) may be varied depending on the first and second microorganisms used. The varying of the conditions to be suitable for the optimal functioning of the microorganisms is within the knowledge of a skilled person.

According to another aspect of the present invention, there is provided a method of producing at least one 3-hydroxybutyrate and/or variants thereof, the method comprising
contacting a recombinant microbial cell according to any aspect of the present invention with a medium comprising a carbon source.

In one example, the method of the present invention may be carried out in an aqueous medium with a pH between 5 and 8, 5.5 and 7. The pressure may be between 1 and 10 bar.

The term “contacting”, as used herein, means bringing about direct contact between the cell according to any aspect of the present invention and the medium comprising the carbon source. For example, the cell, and the medium comprising the carbon source may be in different compartments. On particular, the carbon source may be in a gaseous state and added to the medium comprising the cells according to any aspect of the present invention.

In particular, the aqueous medium may comprise the cells and a carbon source comprising CO and/or CO₂. More in particular, the carbon source comprising CO and/or CO₂ is provided to the aqueous medium comprising the cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

The overall efficiency, alcohol productivity and/or overall carbon capture of the method of the present invention may be dependent on the stoichiometry of the CO₂, CO, and H₂ in the continuous gas flow. The continuous gas flows applied may be of composition CO₂ and H₂. In particular, in the continuous gas flow, concentration range of CO₂ may be about 10-50%, in particular 3% by weight and H₂ would be within 44% to 84%, in particular, 64 to 66.04% by weight. In another example, the continuous gas flow can also comprise inert gases like N₂, up to a N₂ concentration of 50% by weight. Even more in particular, the carbon source according to any aspect of the present invention may comprise 50% or more H₂.

The term ‘about’ as used herein refers to a variation within 20 percent. In particular, the term “about” as used herein refers to +/−20%, more in particular, +/−10%, even more in particular, +/−5% of a given measurement or value.

A skilled person would understand that it may be necessary to monitor the composition and flow rates of the streams. Control of the composition of the stream can be achieved by varying the proportions of the constituent streams to achieve a target or desirable composition. The composition and flow rate of the stream can be monitored by any means known in the art. In one example, the system is adapted to continuously monitor the flow rates and compositions of the streams and combine them to produce a single blended substrate stream in a continuous gas flow of optimal composition, and means for passing the optimised substrate stream to the cell of of the present invention.

Microorganisms which convert CO₂ and/or CO to acetate and/or ethanol, in particular acetate, as well as appropriate procedures and process conditions for carrying out this metabolic reaction is well known in the art. Such processes are, for example described in WO9800558, WO2000014052 and WO2010115054.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli, ATCC1754-Medium may be used in the case of C. ljungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product, 3HB and variants thereof. For example for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. ljungdahlii cell.

According to another aspect of the present invention, there is provided a use of the cell according to any aspect of the present invention for the production of 3-hydroxybutyrate and/or variants thereof.

### BRIEF DESCRIPTION OF FIGURES

FIG. 1 is an illustration of plasmid backbone pSOS95

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Generation of Genetically Modified Acetogens for the Formation of 3HB via Acetoacetate

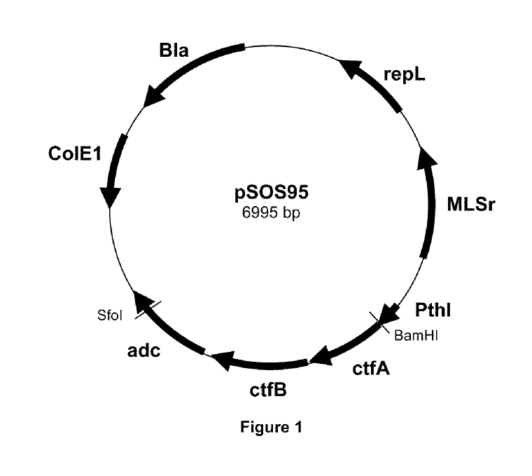

The genes Thiolase (thl) from C. acetobutylicum ATTC 824, Acetoacetat-transferase (ctfAB) from C. acetobutylicum ATTC 824 and the secondary alcohol dehydrogenase (sadh) from C. beijerinckii DSM 6423 were inserted into the vector pEmpty. This plasmid is based on the plasmid backbone pSOS95 (FIG. 1). To use pSOS95, it was digested with BamHI and KasI. This removes the operon ctfA-ctfB-adc, but leaves the thl promoter and the rho-independent terminator of adc. The transformation of C. ljungdahlii and C. autoethanogenum was done as disclosed in Leang et al. 2013. The nucleotide sequences of the enzymes used are SEQ ID NOs:1, 3 (ctfA), 5 (ctfB) and 7 respectively. These sequences were transformed to be controlled by a thiolase promotor and integrated as a single operon into the vector backbone. The created vector was named pTCtS. The vector pTCts was then used to modify C. ljungdahlii and C. autoethanogenum using a method disclosed in Leang et al. 2013. The modified C. ljungdahlii strain was named C. ljungdahlii pTCtS. The modified C. autoethanogenum strain was named C. autoethanogenum pTCtS.

### Example 2

Fermentation of 3HB Strain on H₂ and CO₂ Showing Acetoacetate and 3HB Production.

For cell culture of C. ljungdahlii pTCts 5 mL of the culture were anaerobically grown in 500 ml of medium (ATCC1754 medium: pH 6.0; 20 g/L MES; 1 g/L yeast extract, 0.8 g/L NaCl, 1 g/L NH4Cl, 0.1 g/L KCl, 0.1 g/L KH2PO4, 0.2 g/L MgSO4x 7 H2O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12 ; 50 μg/L p-aminobenzoate; 50 μg/L lipoic acid, approximately 67.5 mg/L NaOH) with about 400 mg/L L-cysteine hydrochloride and 400 mg/L Na₂S×9H₂O, given 100 mg/L erythromycin). Cultivation was carried out in duplicate into 1 L glass bottles with a premixed gas mixture composed of 67% H₂, 33% CO₂ in an open water bath shaker at 37° C., 150 rpm and aeration of 3 L/h for 70.3 h.

The gas entered the medium through a filter with a pore size of 10 microns, which was mounted in the middle of the reactor, at a gassing tube. When sampling each 5 ml sample was removed for determination of OD₆₀₀ nm, pH and the product range. The determination of the product concentration was performed by semi-quantitative 1 H-NMR spectroscopy. As an internal quantification standard sodium trimethylsilylpropionate was used (T(M) SP). A culture produced 7 ppm acetoacetate and 26 ppm 3-HB in 70.3 hours. The other culture produced 24 ppm acetoacetate and 104 ppm 3-HB in 70.3 hours.

### Example 3

Fermentation of Vector Control Strain with No Production of Acetoacetate or 3HB

For cell culture of C. ljungdahlii pEmpty 5 mL of the culture were anaerobically grown in 500 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO_{4,} 0.2 g/L MgSO₄×7H₂O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12 ; 50 μg/L p-aminobenzoate; 50 μg/L lipoic acid, approximately 67.5 mg/L NaOH) with about 400 mg/L L-cysteine hydrochloride and 400 mg/L Na₂S×9H₂O, given 100 mg/L erythromycin). Cultivation was carried out in duplicate into 1 L glass bottles with a premixed gas mixture composed of 67% H₂, 33% CO₂ in an open water bath shaker at 37° C., 150 rpm and aeration of 3 L/h for 70.3 h.

The gas entered the medium through a filter with a pore size of 10 microns, which was mounted in the middle of the reactor, at a gassing tube. When sampling each 5 ml sample was removed for determination of OD₆₀₀ nm, pH and the product range. The determination of the product concentration was performed by semi-quantitative 1 H-NMR spectroscopy. As an internal quantification standard sodium trimethylsilylpropionate was used (T(M) SP). Neither acetoacetate nor 3HB was produced.

### Example 4

Fermentation of C. autoethanogenum pTCtS on CO, H₂ and CO₂ Showing 3HB Production.

For cell culture of C. autoethanogenum pTCts 5 mL of the culture was anaerobically grown in 500 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO₄, 0.2 g/L MgSO₄×7H₂O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12; 50 μg/L p-aminobenzoate; 50 μg/L lipoic acid, approximately 67.5 mg/L NaOH) with about 400 mg/L L-cysteine hydrochloride and 400 mg/L Na₂S×9H₂O, given 100 mg/L erythromycin). Cultivation was carried out in duplicate into 1 L glass bottles with a premixed gas mixture composed of 55% CO, 20% Hz, 10% CO₂ and 15% N₂ in an open water bath shaker at 37° C., 150 rpm and aeration of 3 L/h for 7 days.

The gas entered the medium through a filter with a pore size of 10 microns, which was mounted in the middle of the reactor, in a gassing tube. When sampling, 5 ml samples were removed for determination of OD₆₀₀, pH and the product range. The determination of the product concentration was performed by semi-quantitative 1 H-NMR spectroscopy. As an internal quantification standard sodium trimethylsilylpropionate was used (T(M) SP). The modified strain produced 40 ppm 3-HB in 7 days.

### Example 5

Fermentation of C. autoethanogenum Vector Control Strain with No Production of Acetoacetate or 3HB

For cell culture of C. autoethanogenum pEmpty 5 mL of the culture was anaerobically grown in 500 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO_{4,} 0.2 g/L MgSO₄×7H₂O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12 ; 50 μg/L p-aminobenzoate; 50 μg/L lipoic acid, approximately 67.5 mg/L NaOH) with about 400 mg/L L-cysteine hydrochloride and 400 mg/L Na₂S×9H₂O, given 100 mg/L erythromycin). Cultivation was carried out in duplicate in 1 L glass bottles with a premixed gas mixture composed of 55% CO, 20% Hz, 10% CO₂ an open water bath shaker at 37° C., 150 rpm and aeration of 3 L/h for 7 days.

The gas entered the medium through a filter with a pore size of 10 microns, which was mounted in the middle of the reactor, in a gassing tube. When sampling, 5 ml samples were removed for determination of OD₆₀₀ nm, pH and the product range. The determination of the product concentration was performed by semi-quantitative 1 H-NMR spectroscopy. As an internal quantification standard sodium trimethylsilylpropionate was used (T(M) SP). Neither acetoacetate nor 3HB was produced.

### REFERENCES

Altschul S. et al., 1990, BLAST Manual,
Atsumi et al., Nature, 2008 451.7174:86-89
Barker et al., J. Bacteriol. 1982, 152(I):201-7
Barker et al., Biol. Chem. 1978, 253(4): 1219-25
Charrier C., Microbiology, 2006, 152: 179-185
de la Plaza et al FEMS Microbiol Lett. 2004 238: 367-374
Deveroy, J. et al., Nucleic Acid Research 12 (1984), Seite 387, Genetics Computer Group University of Wisconsin, Medicine (Wi)
Hanai et al., Appl Environ Microbiol, 2007, 73:7814-7818
Ismail et al. J Bacteriol 1993, 175: 5097-5105
Jeon et al., Biotechnol. 2008, 135.2:127-133
Jojima et al., Appl Microbiol Biotechnol, 2008 77: 1219-1224
Kapatral et al., Bact. 2002, 184(7) 2005-2018
Khorkin et a, J Mol Biol. 1998, 22: 278(5): 967-981
Kinoshita et al., Appl. Microbiol. Biotchenol. 1985, 22:249-254
Kosaka et al., Bio sci. Biotechnol Biochem. 2007, 71:58-68
Kreimeyer, et al., Biol. Chem. 2007, 282 (10) 7191-7197
Martin et al, Nat. Biotechnol. 2003, 21.7:796-802
Perez et al., Biol. Chem. 2008, 283.12:7346-7353
Peterson and Bennet, Appl Environ Microbiol. 1990 56: 3491-3498
Riviere et al., Biol. Chem. 2004, 279:45337-45346
Seedorf et al., Proc. Natl. Acad. Sci. USA 2008, 105:2128-2133
Sohling and Gottschalk, J Bacteriol, 1996 178:871-880
Sulzenbacher et al., Mol. Biol. 2004, 342.2:489-502
Tani et al., Appl. Environ. Microbiol. 2000, 66.12:5231-5335
van Grinsven et al., J. Biol. Chem. 2008 283: 1411-1418
Walter et al., Bacteriol. 1992, 174.22:7149;7158
Wiesenborn et al Appl Environ Microbiol. 1988, 54: 2717-2722
Wiesenborn et al., Appl Environ Microbiol. 1989, 55:323-9
WO9800558, WO2000014052, WO2010115054.

1. A microbial cell which is capable of producing acetoacetate, 3-hydroxybutyrate and/or 3-hydroxybutyrate variants, wherein the cell is genetically modified to comprise an increased expression relative to its wild type cell of:
an enzyme E₁ capable of catalysing the conversion of acetyl-CoA to acetoacetyl-CoA;
an enzyme E₂ capable of catalysing the conversion of acetoacetyl-CoA to acetoacetate; and
an enzyme E₃ capable of catalysing the conversion of acetoacetate to 3-hydroxybutyrate and/or variants thereof and wherein the genetically modified cell has reduced or no expression of acetoacetate decarboxylase (Adc; EC 4.1.1.4).

2. The cell according to claim 1, wherein E₁ is a thiolase, E₂ is an acetoacetate CoA transferase and/or E₃ is a secondary alcohol dehydrogenase.

3. The cell according to claim 1, wherein the thiolase (E₁) is from C. acetobutylicum.

4. The cell according to claim 1, wherein the acetoacetate CoA transferase (E₂) is from C. acetobutylicum,

5. The cell according to claim 1, wherein the secondary alcohol dehydrogenase (E₃) is from C. beijerinckii.

6. The cell according to claim 1, wherein
E₁ comprises 60% sequence identity with SEQ ID NO: 2,
E₂ comprises 60% sequence identity with SEQ ID NO: 4 or 6 and/or
E₃ comprises 60% sequence identity with SEQ ID NO: 8.

7. The cell according to claim 1, wherein
E₁ comprises SEQ ID NO: 2,
E₂ comprises SEQ ID NO: 4 and 6 and
E₃ comprises SEQ ID NO: 8.

8. The cell according to claim 1, wherein the cell is an acetogenic cell.

9. The cell according to claim 8, wherein the acetogenic cell is selected from the group consisting of Clostridium autothenogenum DSMZ 19630, Clostridium ragsdahlei ATCC no. BAA-622, Clostridium autoethanogenum, Moorella sp HUC22-1, Moorella thermoaceticum, Moorella thermoautotrophica, Rumicoccus productus, Acetoanaeroburn, Oxobacter pfennigii, Methanosarcina barkeri, Methanosarcina acetivorans, Carboxydothermus, Desulfotomaculum kutznetsovii, Pyrococcus, Peptostreptococcus, Butyribacterium methylotrophicum ATCC 33266, Clostridium formicoaceticum, Clostridium butyricum, Lactobacillus deibrukiis, Propionibacterium acidoproprionici, Proprionispera arboris, Anaerobierspirillum succiniproducens, Bacterioides amylophilus, Becterioides ruminicola, Thermoanaerobacter kivui, Acetobacterium woodii, Acetoanaerobium notera, Clostridium aceticum, Butyribacterium methylotrophicum, Moorella thermoacetica, Eubacterium limosum, Peptostreptococcus productus, Clostridium ljungdahlii, Clostridium ATCC 29797 and Clostridium carboxidivorans.

10. The cell according to claim 1, wherein the cell is Clostridium ljungdahlii or Clostridium autothenogenum DSMZ 10061.

11. A method of producing acetoacetate, 3-hydroxybutyrate and/or 3-hydroxybutyrate variants, the method comprising
contacting a recombinant microbial cell according to claim 1 with a medium comprising a carbon source.

12. The method according to claim 11, wherein the carbon source comprises CO₂ and/or CO.

13. The method according to claim 11, wherein the carbon source comprises 50% or more H₂.

14. Use of the cell according to claim 1 for the production of acetoacetate, 3-hydroxybutyrate and/or 3-hydroxybutyrate variants.
